(19) United States
(12) Plant Patent Application Publication
(10) Pub. No.: US 2002/0138886 P1
(43) Pub. Date: Sep. 26, 2002

(54) SOLIDAGO PLANT NAMED 'DANSOLMONTE'

(76) Inventor: GABRIEL DANZIGER, NIR-ZVI (IL)

Correspondence Address: FOLEY & LARDNER, SUITE 500, 3000 K STREET NW, P O BOX 25696, WASHINGTON, DC, 200078696

(21) Appl. No.: 09/323,146

(22) Filed: Jun. 1, 1999

(51) Int. Cl. A01H 5/00

(52) U.S. Cl. PLT/263

(57) ABSTRACT

A new and distinct Solidago plant named ‘Dansolmonte’ characterized by having small yellow flowers; rapid growth cycle; and an even growth rate of the plant.

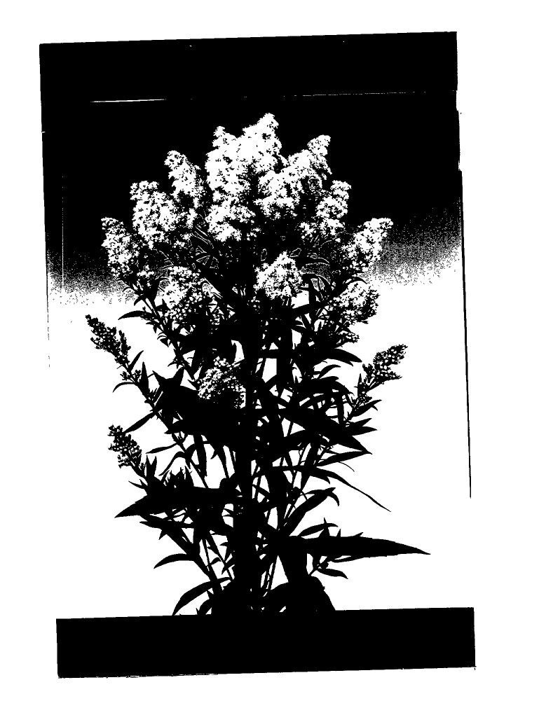

### BACKGROUND OF THE INVENTION

The present invention relates to a new and distinct cultivar of goldenrod plant, botanically known as Solidago L. and hereinafter referred to by the cultivar name ‘Dansolmonte’.

Goldenrods are members of the sunflower family (Asteraceae). There are approximately 100 species of Solidago plants found in North America, most of which are geographically located in the East. Goldenrods are perennial plants that commonly grow along streambanks, ditches, roadsides and other areas where the soil is moist and rich.

The new cultivar was originated from a cross made in a controlled breeding program in Mishmar Hashiva, Israel. The female parent is a Solidago cultivar designated as a seedling from the breeding program. The male parent is an unknown Solidago cultivar. ‘Dansolmonte’ was discovered and selected by the inventor, Gabriel Danziger, as a flowering plant within the progeny of the stated cross in a controlled environment in Mishmar Hashiva, Israel.

Asexual reproduction of the new cultivar by leaf-cutting was first performed by the inventor at Mishmar Hashiva, Israel and has demonstrated that the combination of characteristics as herein disclosed for the new cultivar are firmly fixed and retained through successive generations of asexual reproduction.

### BRIEF DESCRIPTION OF THE INVENTION

The following traits have been repeatedly observed and are determined to be basic characteristics of ‘Dansolmonte’ which in combination distinguish this Solidago as a new and distinct cultivar:

1. small yellow flowers

2. rapid growth cycle; and

3. an even growth rate of the plant.

‘Dansolmonte’ has not been observed under all possible environmental conditions. The phenotype of the new cultivar may vary significantly with variations in environment such as temperature, light intensity, and daylength without any change in the genotype of the plant. The following observations, measurements and values describe the new cultivar as grown in Mishmar Hashiva, Israel under conditions which closely approximate those generally used in commercial practice.

Of the many commercial cultivars known to the present inventor, the most similar in comparison to ‘Dansolmonte’ is the well-known Solidago cultivar ‘Tara’. In comparison to ‘Tara’, ‘Dansolmonte’ has flowers of similar size and color, but the stems of ‘Dansolmonte’ are more stable. The side-stems of ‘Dansolmonte’ are longer and more upright than ‘Tara’.

### BRIEF DESCRIPTION OF THE DRAWING

The accompanying photographic illustration shows a typical plant of ‘Dansolmonte’ following growth under appropriate growing conditions, with colors being as true as possible with illustrations of this type.

### DETAILED BOTANICAL DESCRIPTION

The following observations, measurements and values describe the new cultivar as grown in Mishmar Hashiva, Israel, under conditions which closely approximate those generally used in commercial practice. Color references are made to The Royal Horticultural Society Colour Chart (RHS), except where general colors of ordinary significance are used. Color values were taken under daylight conditions in the morning in Mishmar Hashiva, Israel.

Origin: Mishmar Hashiva, Israel

Parentage:

Male parent.—Unknown Solidago cultivar.

Female parent.—Seedling from breeding program.

Classification:

Botanical.—Solidago L.

Commercial.—Goldenrod, cv. ‘Dansolmonte’.

Propagation: By leaf-cutting.

Plant:

Growth habit.—Panicle inflorescence with approximately 7-10 stems. After pinching, the height of the branches are approximately 70-100 cm. It has a main branch with side branches having small, yellow flowers, and many buds on each branch.

Foliage:

Shape.—Linear, serrated.

Color.—Dark-green (RHS 137C).

Inflorescence:

Capitulum.—Form: Elongated Type: Panicle, one-sided Diameter across face: Approximately 6.5 cm.

Ray florets.—Color (upper surface): Yellow (RHS 9B) Color (under surface): Yellow (RHS 5A) Shape: One row of petals.

Disc florets.—Color (mature): Yellow (RHS 9A) Diameter: Approximately 0.1-0.2 cm.

I claim:

1. A new and distinct Solidago plant named ‘Dansolmonte’, substantially as illustrated and described herein.
